(19) United States
(12) Patent Application Publication
(10) Pub. No.: US 2012/0101384 A1
(43) Pub. Date: Apr. 26, 2012

(54) ULTRASONIC DIAGNOSTIC DEVICE

(75) Inventor: Manabu Migita, Kanagawa (JP)

(73) Assignee: PANASONIC CORPORATION, Osaka (JP)

(21) Appl. No.: 13/380,601

(22) PCT Filed: Sep. 24, 2010
(86) PCT No.: PCT/JP2010/005775

§ 371 Date: Dec. 23, 2011

(30) Foreign Application Priority Data
Sep. 28, 2009 (JP) 2009-223204
Sep. 28, 2009 (JP) 2009-223205

(51) Int. Cl. A61B 8/14

(52) U.S. Cl. 600/443

(57) ABSTRACT

An ultrasonic diagnostic apparatus according to the present invention includes: a transmitting and receiving section, which drives a probe repeatedly to send out ultrasonic waves toward a subject and which makes the probe receive reflected echoes produced by having the ultrasonic waves reflected by the subject, thereby generating received signals; a color flow mapping signal processing section for sequentially generating, based on the received signals, blood flow velocity data about a portion of each frame representing the blood flow of the subject; a persistence processing section for performing persistence processing on the blood flow velocity data of each frame; a tomographic image signal processing section for generating B-mode tomographic image frame data based on the received signals; and an image synthesizing section for synthesizing together the persistence-processed blood flow velocity data and the B-mode tomographic image frame data. The persistence processing section makes an aliasing decision based on the blood flow velocity data of the current frame and the persistence-processed blood flow velocity data of an earlier frame that precedes the current frame, and changes a persistence coefficient dynamically according to a result of the aliasing decision and based on those blood flow velocity data of the current and earlier frames.

### TECHNICAL FIELD

The present invention relates to an ultrasonic diagnostic apparatus and more particularly relates to a technique for processing a persistence involved with color flow mapping.

### BACKGROUND ART

An ultrasonic diagnostic apparatus sends out an ultrasonic wave toward a subject, receives its reflected echo, and then analyzes information included in the echo, thereby generating an image representing the subject's internal body tissue. For example, blood flowing through a subject's internal body tissue can also be represented as an image by so-called “color flow mapping” (which will be sometimes abbreviated herein as “CFM”). And ultrasonic diagnostic apparatuses that can indicate the blood flow status are used extensively in the entire field of medical treatment in general.

Color flow mapping is also called “color Doppler imaging (CDI)”, which uses the Doppler effect. When blood flow is irradiated with an ultrasonic wave, its reflected echo will have a Doppler shift, of which the magnitude changes according to the velocity of the blood flow, due to the Doppler effect. By collecting information about the Doppler shift by orthogonal detection and subjecting that information to high-pass filtering using a moving target indicator (MTI) filter, autocorrelation processing and noise reduction processing, information about the blood flow velocity can be obtained. And if the blood flow velocity information thus obtained is transformed into color information to be superimposed two-dimensionally on a B-mode tomographic image, the status of the blood flow inside the subject's body can be seen by the user.

The intensity of a received signal representing the echo that has been reflected from blood flow is much less than that of a received signal representing the echo that has been reflected from a scatterer or boundary of a tissue of interest for use to generate a B-mode tomographic image. For that reason, the blood flow velocity and the blood flow power (representing the rate of blood flowing) to be obtained by color flow mapping signal processing tend to lose their stability.

Particularly if the region of interest has a low blood flow velocity or is a peripheral vessel, the blood flow power decreases so much that information about the blood flow velocity or the blood flow power tends be lost very easily during noise reduction processing to cut down only system noise or acoustic noise. As a result, the resultant blood flow image will have a blackout portion where the blood flow should be displayed. For example, if the blood flow inside a subject's body needs to be displayed as an image at a rate of several to several tens of frames per second, the blow flow portion will be blacked out in some of those frames. In that case, the blood flow portion will suddenly disappear from the tomographic image, thus decreasing the smoothness of the image or making the viewer sense unnaturalness.

To overcome such a problem, a conventional ultrasonic diagnostic apparatus that adopts a color flow mapping technique usually makes temporal interpolation called “persistence processing” in a late stage of signal processing. Hereinafter, it will be described how the persistence processing is carried out according to the conventional color flow mapping technique disclosed in Patent Document No. 1.

In the conventional ultrasonic diagnostic apparatus shown in FIG. 7, an ultrasonic wave transmitting and receiving section 402 drives a probe 401, thereby sending out an ultrasonic wave toward a subject, and also makes the probe 401 receive its reflected echo that has been produced by a subject, thereby generating a received signal. If a B-mode tomographic image needs to be generated, the ultrasonic wave transmitting and receiving section 402 adopts a best ultrasonic wave transmitting and receiving mode to generate a B-mode tomographic image, and outputs a received signal obtained to a tomographic image signal processing section 409. On the other hand, if a color flow mapping tomographic image needs to be generated, the ultrasonic wave transmitting and receiving section 402 adopts a best ultrasonic wave transmitting and receiving mode to generate a color flow mapping tomographic image, and outputs a received signal obtained to a color flow mapping signal processing section (which will be referred to herein as a “CFM signal processing section”) 403. Generally speaking, when a color flow mapping tomographic image needs to be generated, the ultrasonic wave transmitting and receiving section 402 transmits and receives ultrasonic waves a number of times on the same acoustic line in order to obtain a stabilized color flow mapping tomographic image.

The CFM signal processing section 403 performs orthogonal detection, MTI filtering and autocorrelation processing on the received signal, calculates the blood flow velocity and the blood flow power, carries out noise reduction processing to cut down system noise or acoustic noise, and then outputs the blood flow velocity and the blood flow power to a frame memory section 404.

The frame memory section 404 is implemented as a ring buffer and stores the blood flow velocities and blood flow powers of the current through N^{th} latest frames (where N an integer that is equal to or greater than one) on a frame-by-frame basis. As used herein, the “frame” refers to a group of blood flow velocity data and a group of blood flow power data that form one picture of CFM tomographic image.

A frame memory choosing section 405 instructs the frame memory section 404 to choose a number of CFM frames specified in advance from the frame memory section 404 and output the data of those frames to a persistence computation section 407. Based on the CFM frame data retrieved from the frame memory section 404 and a persistence coefficient provided by a persistence coefficient setting section 406, the persistence computation section 407 carries out persistence computation and outputs the result to a CFM DSC (digital scan converter) section 408. The persistence computation is a simple weighting operation. And the persistence coefficient provided by the persistence coefficient setting section 406 is a fixed coefficient that has been set in advance by the system.

The CFM DSC section 408 converts the CFM frame data that has been provided by the persistence computation section 407 and then outputs the converted data to an image synthesizing section 411.

The tomographic image signal processing section 409 cuts down unwanted noise of the received signal by subjecting it to dynamic filtering and then subjects the received signal to envelope detection processing and dynamic range compression processing, thereby outputting tomographic image frame data to a tomographic image DSC section 410. In response, the tomographic image DSC section 410 converts the coordinates of the tomographic image frame data that has been provided by the tomographic image signal processing section 409 and then outputs the converted data to the image synthesizing section 411.

The image synthesizing section 411 synthesizes together the frame data that has been provided by the CFM DSC section 410 and the tomographic image DSC section 410 on a pixel-by-pixel basis, thereby generating synthesized image frame data. Specifically, the image synthesizing section 411 synthesizes together the two data either on a pixel-by-pixel basis or on the basis of each pair of associated measuring points so that if the blood flow velocity is zero, tomographic image frame data is displayed but that the CFM frame data is displayed otherwise. Also, the image synthesizing section 411 transforms the data into color information according to the blood flow velocity or blood flowing direction and then outputs the color information thus obtained to a display section 412, which displays the data provided by the image synthesizing section 411.

### CITATION LIST

### Patent Literature

Patent Document No. 1: Japanese Patent Application Laid-Open Publication No. 2-286140

### SUMMARY OF INVENTION

### Technical Problem

The conventional ultrasonic diagnostic apparatus performs the persistence processing in order to prevent the output of the CFM signal processing section 403 from losing stability to cause a blackout on the blood flow displayed on the monitor due to a low blood flow velocity or a low blood flow power and due to their instability. Specifically, by using a persistence coefficient with a higher priority given to the past frame data rather than the current frame data being obtained by scanning, a persistence effect is produced, thereby minimizing generation of such a blackout in the image.

Such a method, however, does not always work fine in inspecting an artery with significantly changing blood flow velocities. For example, the blood flowing through a carotid artery changes its velocity steeply depending on whether the heart is now dilating or contracting. Specifically, the blood flow increases its velocity in the contraction phase for a very short time with respect to one cardiac cycle and decreases its velocity in the diastolic phase. On top of that, the difference between the maximum and minimum blood flow velocities is greater than in any other region to inspect. As a result, in the diastolic phase, the blood flow velocity at the carotid artery remains low for a relatively long time with respect to one cardiac cycle and the CFM signal processing section 403 loses its stability.

To prevent such a blackout from being produced on a blood flow image being displayed, the persistence coefficient is preferably defined so as to increase the persistence effect through the persistence processing. Then, even if the blood flow velocity is low, a moving picture can still be displayed smoothly without causing any blackout. In that case, however, no blood flow with high velocities can be displayed in the contraction phase.

On top of that, the persistence processing performed by conventional ultrasonic diagnostic apparatuses does not always work fine when a peripheral blood vessel needs to be inspected. For example, a thyroid, a liver, a kidney, and other organs have a peripheral vessel branching from the main blood vessel. In inspecting any of these organs, it is very important to understand what structure the peripheral blood vessel has.

The variation in the rate of blood flowing through a peripheral vessel with time is relatively moderate. However, as that blood vessel is physically thin, its blood flow power tends to be much lower than that of the carotid artery or the heart. Consequently, since the blood flow power is low, the Doppler shift will lose its stability so much that the output of the CFM signal processing section 403 will eventually lack stability, too.

That is why unless the persistence processing is carried out, the peripheral blood vessel will be sometimes visible but sometimes invisible on the tomographic image, thus making it difficult for the viewer to track it on the moving picture. On the other hand, if the persistence processing is carried out, the blood flowing through the peripheral blood vessel will be smoothed out too much on the tomographic image with time to keep the peripheral blood vessel visible through the persistence processing. In that case, the peripheral blood vessel can be detected much less accurately.

It is therefore an object of the present invention to provide an ultrasonic diagnostic apparatus that can accurately sense any variation in blood flow even at any region to inspect where the blood flow velocity changes significantly (such as a carotid artery) and that can display a blood flow moving picture smoothly without causing any blackout even at a low blood flow velocity. Another object of the present invention is to provide an ultrasonic diagnostic apparatus that can display a moving picture on which even a blood vessel portion with low blood flow power (such as a peripheral blood vessel) is easily trackable.

### Solution to Problem

An ultrasonic diagnostic apparatus according to the present invention includes: a transmitting and receiving section, which drives a probe a number of times to send out ultrasonic waves toward a subject and which makes the probe receive reflected echoes that have been produced by having the ultrasonic waves reflected by the subject, thereby generating multiple received signals one after another; a color flow mapping signal processing section for sequentially generating, based on the received signals, blood flow velocity data about a portion of each frame representing the blood flow of the subject; a persistence processing section for performing persistence processing on the blood flow velocity data of each frame; a tomographic image signal processing section for generating B-mode tomographic image frame data based on the received signals; and an image synthesizing section for synthesizing together the persistence-processed blood flow velocity data and the B-mode tomographic image frame data. The persistence processing section makes an aliasing decision based on the blood flow velocity data of the current frame and the persistence-processed blood flow velocity data of an earlier frame that precedes the current frame, and changes a persistence coefficient dynamically according to a result of the aliasing decision and based on those blood flow velocity data of the current and earlier frames.

In one preferred embodiment, the ultrasonic diagnostic apparatus includes: a transmitting and receiving section, which drives a probe a number of times to send out ultrasonic waves toward a subject and which makes the probe receive reflected echoes that have been produced by having the ultrasonic waves reflected by the subject, thereby generating multiple received signals one after another; a color flow mapping signal processing section for sequentially generating, based on the received signals, blood flow velocity data about a portion of each frame representing the blood flow of the subject; a persistence processing section for performing persistence processing on the blood flow velocity data of each frame; a tomographic image signal processing section for generating B-mode tomographic image frame data based on the received signals; and an image synthesizing section for synthesizing together the persistence-processed blood flow velocity data and the B-mode tomographic image frame data. The persistence processing section includes: a first memory section for storing the blood flow velocity data of the current frame; a second memory section for storing the persistence-processed blood flow velocity data of the earlier frame that precedes the current frame; an aliasing decision section for making an aliasing decision by retrieving the respective blood flow velocity data from the first and second memory sections; a persistence coefficient determining section for determining the persistence coefficient based on a result of the aliasing decision and on the blood flow velocity data that is stored in the first memory section; and a persistence computation section for performing a persistence computation on the blood flow velocity data that is stored in the first memory section using the persistence coefficient based on the result of the aliasing decision and outputting a result of the computation as the persistence-processed blood flow velocity data.

In this particular preferred embodiment, by comparing the respective blood flow velocity data that are stored in the first and second memory sections to multiple threshold values, the aliasing decision section determines whether or not aliasing has occurred and whether or not the blood flow velocity data of the current frame is in an aliasing region.

In a specific preferred embodiment, the persistence processing section further includes a third memory section that stores a table of reference including persistence coefficients with two or more different values that are associated with the blood flow velocity values.

In a more specific preferred embodiment, in the table of reference, a persistence coefficient with a constant value is associated with blood flow velocities, of which the values are equal to or greater than a predetermined value.

In yet another preferred embodiment, the ultrasonic diagnostic apparatus includes: a transmitting and receiving section, which drives a probe a number of times to send out ultrasonic waves toward a subject and which makes the probe receive reflected echoes that have been produced by having the ultrasonic waves reflected by the subject, thereby generating multiple received signals one after another; a color flow mapping signal processing section for sequentially generating, based on the received signals, blood flow velocity data about a portion of each frame representing the blood flow of the subject; a persistence processing section for performing persistence processing on the blood flow velocity data of each frame; a tomographic image signal processing section for generating B-mode tomographic image frame data based on the received signals; and an image synthesizing section for synthesizing together the persistence-processed blood flow velocity data and the B-mode tomographic image frame data. The persistence processing section includes: a first memory section for storing the blood flow velocity data of the current frame; a second memory section for storing the persistence-processed blood flow velocity data of the earlier frame that precedes the current frame; an aliasing decision section for making an aliasing decision by retrieving the respective blood flow velocity data from the first and second memory sections; a first persistence coefficient determining section for determining a first persistence coefficient based on a result of the aliasing decision and on the blood flow velocity data that is stored in the first memory section; a first persistence computation section for performing a persistence computation on the blood flow velocity data that is stored in the first memory section using the first persistence coefficient based on the result of the aliasing decision; a second persistence coefficient determining section for determining a second persistence coefficient based on the result of the aliasing decision and on the blood flow velocity data that is stored in the second memory section; a second persistence computation section for performing a persistence computation on the blood flow velocity data that is stored in the first memory section using the second persistence coefficient based on the result of the aliasing decision; and a maximum value choosing section for comparing the two absolute values of the computational results provided by the first and second persistence computation sections to each other and outputting the greater one of the two absolute values as the persistence-processed blood flow velocity data.

In this particular preferred embodiment, by comparing the respective blood flow velocity data that are stored in the first and second memory sections to multiple threshold values, the aliasing decision section determines whether or not aliasing has occurred and whether or not the blood flow velocity data of the current frame is in an aliasing region.

In another preferred embodiment, the persistence processing section further includes: a third memory section that stores a first table of reference including a first set of persistence coefficients with two or more different values that are associated with the blood flow velocity values; and a fourth memory section that stores a second table of reference including a second set of persistence coefficients with two or more different values that are associated with the blood flow velocity values.

In a specific preferred embodiment, even if one of the persistence coefficients of the first set and one of the persistence coefficients of the second set, which are stored in the first and second tables of reference, respectively, are associated with the same blood flow velocity value, those two persistence coefficients have mutually different values.

In another preferred embodiment, in the first table of reference, a persistence coefficient with a constant value is associated with blood flow velocities, of which the values are equal to or greater than a predetermined value.

In yet another preferred embodiment, the persistence-processed blood flow velocity data of the earlier frame that precedes the current frame belongs to the previous frame.

### Advantageous Effects of Invention

According to the present invention, an aliasing decision is made based on the respective blood flow velocity data of the current frame and an earlier frame that precedes the current frame, and changes a persistence coefficient dynamically according to a result of the aliasing decision and based on the blood flow velocity data of the current frame. Consequently, the present invention provides an ultrasonic diagnostic apparatus that can accurately sense any variation in blood flow and that can display a blood flow moving picture smoothly without causing any blackout even at a low blood flow velocity.

In addition, according to the present invention, two persistence-processed blood flow velocities are obtained using two persistence coefficients that have been determined based on the respective blood flow velocity data of the current and earlier frames, and one of the two blood flow velocities that has the greater absolute value is chosen and used to display a blood, flow image. As a result, even the blood flowing through a peripheral blood vessel, where the blood flow power will often lose its stability, can be displayed constantly without flickering. And a blood flow moving picture can be displayed consistently without letting an overly smoothed blood, flowing through the peripheral blood vessel, disappear from the screen.

### BRIEF DESCRIPTION OF DRAWINGS

FIG. 1 is a block diagram illustrating a first preferred embodiment of an ultrasonic diagnostic apparatus according to the present invention.

FIGS. 2(a) and 2(b) are schematic representations showing how to make an aliasing decision when a persistence computation is performed on blood flow velocity data using a persistence coefficient according to the first preferred embodiment.

FIG. 3(a) is a schematic representation showing how to make an aliasing decision according to the first preferred embodiment, and FIG. 3(b) is a graph showing the relation satisfied by the data of a table of reference.

FIG. 4 is a block diagram illustrating a second preferred embodiment of an ultrasonic diagnostic apparatus according to the present invention.

FIGS. 5(a) and 5(b) are schematic representations showing how to make an aliasing decision when a persistence computation is performed on blood flow velocity data using a persistence coefficient according to the second preferred embodiment.

FIG. 6(a) is a schematic representation showing how to make an aliasing decision according to the second preferred embodiment, and FIGS. 6(b) and 6(c) are graphs showing the relations satisfied by the data of first and second tables of reference.

FIG. 7 is a block diagram illustrating a conventional ultrasonic diagnostic apparatus.

### DESCRIPTION OF EMBODIMENTS

### Embodiment 1

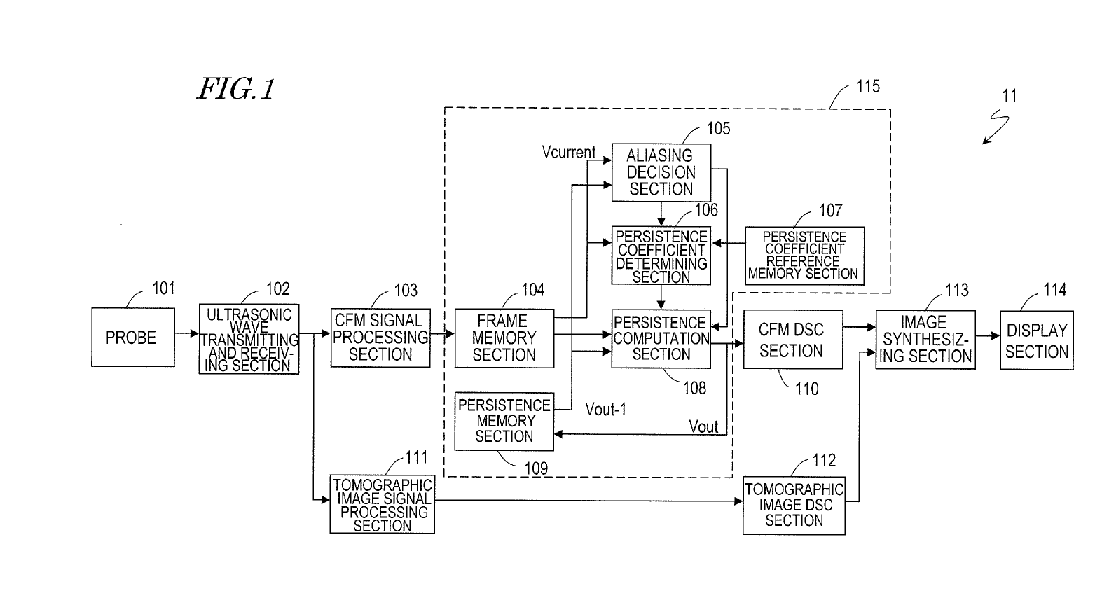

Hereinafter, a first preferred embodiment of an ultrasonic diagnostic apparatus according to the present invention will be described with reference to the accompanying drawings. FIG. 1 is a block diagram illustrating a first preferred embodiment of an ultrasonic diagnostic apparatus according to the present invention. The ultrasonic diagnostic apparatus 11 shown in FIG. 1 includes a probe 101, an ultrasonic wave transmitting and receiving section 102, a CFM signal processing section 103, a persistence processing 115, a tomographic image signal processing section 111, a CFM DSC section 110, a tomographic image DSC section 112, an image synthesizing section 113, and a display section 114. Among these components, the probe 101 and the display section 114 may be general-purpose ones and may be omitted from this ultrasonic diagnostic apparatus 11.

The ultrasonic wave transmitting and receiving section 102 generates a drive signal to drive the probe 101 and outputs the signal to the probe 101, thereby sending out an ultrasonic wave from the probe 101 toward a subject. Also, the ultrasonic wave transmitting and receiving section 102 makes the probe 101 receive reflected echoes, which have been produced by having the transmitted ultrasonic wave reflected by the subject, thereby generating a received signal. More specifically, the probe 101 is made up of multiple piezoelectric transducers. And this ultrasonic wave transmitting and receiving section 102 drives the probe 101 while controlling the delays caused by those piezoelectric transducers so that the ultrasonic wave sent out by each of those piezoelectric transducers defines a single ultrasonic beam and that the subject is scanned with a number of such ultrasonic beams. Those reflected echoes are received by the respective piezoelectric transducers and the ultrasonic wave transmitting and receiving section 102 controls the delays caused by the respective piezoelectric transducers, thereby generating received signals that are associated with the respective ultrasonic beams transmitted. In this case, every time the subject is scanned with an ultrasonic beam, data for one frame can be obtained. And by repeatedly transmitting and receiving ultrasonic waves several to several tens of times a second, several to several tens of frames of received signals are generated one after another per second.

The ultrasonic diagnostic apparatus 11 of this preferred embodiment generates a B-mode tomographic image and a color flow mapping image, synthesizes these two images together, and then displays the synthetic image on the display section 114. Thus, the ultrasonic wave transmitting and receiving section 102 transmits and receives ultrasonic waves both in order to generate the B-mode tomographic image and to generate the color flow mapping image. The B-mode tomographic image and the color flow mapping image may or may not be displayed at the same frame rate (i.e., may or may not have the same number of frames to display per second). If these two kinds of images have the same frame rate, ultrasonic waves may be alternately transmitted and received to generate a B-mode tomographic image and to generate a color flow mapping image.

In generating a B-mode tomographic image, the ultrasonic wave transmitting and receiving section 102 controls its mode of transmitting and receiving ultrasonic waves appropriately so as to generate the B-mode tomographic image as intended and outputs a received signal obtained to the tomographic image signal processing section 111. On the other hand, in generating a color flow mapping tomographic image, the ultrasonic wave transmitting and receiving section 102 controls its mode of transmitting and receiving ultrasonic waves appropriately so as to generate the color flow mapping tomographic image as intended and outputs a received signal obtained to the CFM signal processing section 103. In general, when a color flow mapping tomographic image needs to be generated, the ultrasonic wave transmitting and receiving section 102 repeatedly transmits and receives ultrasonic waves a number of times along the same acoustic line to generate a color flow mapping tomographic image with good stability.

The CFM signal processing section 103 performs orthogonal detection processing, MTI filtering and autocorrelation processing on the received signal to calculate a blood flow velocity and a blood flow power, and then carries out noise reduction processing to cut down either system noise or acoustic noise. The CFM frame data includes at least blood flow velocity data but may also include blood flow power data and blood flow velocity variance data as well. The CFM signal processing section 103 repeatedly performs this processing on each of the received signals that form respective frames one after another. The CFM frame data generated by the CFM signal processing section 103 is output to the persistence processing section 115 on a frame-by-frame basis.

The persistence processing section 115 performs persistence processing On the CFM frame data on a frame-by-frame basis using a persistence coefficient. The ultrasonic diagnostic apparatus 11 of this preferred embodiment determines the persistence coefficient by the blood flow velocity of the current frame. That is to say, the persistence coefficient is not a constant value but is a dynamic value that is variable according to the blood flow velocity of the current frame. Consequently, the persistence coefficient can be changed, and the persistence effect can be controlled, according to the blood flow velocity. To present the blood flow as a moving picture, however, ultrasonic waves need to be transmitted and received with a pulse Doppler system. That is why the measurable blood flow velocity is restricted by the pulse repetition frequency (PRF). As a result, aliasing will occur in the blood flow velocity, thus making it difficult to estimate the blood flow velocity accurately.

To determine whether aliasing has occurred or not, the ultrasonic diagnostic apparatus 11 of this preferred embodiment uses the respective blood flow velocity data of the current frame and the previous frame. For that purpose, the persistence processing section 115 includes a frame memory section (corresponding to the “first memory section”) 104, an aliasing decision section 105, a persistence coefficient determining section 106, a persistence coefficient reference memory section (corresponding to the “third memory section”) 107, a persistence computation section 108, and a persistence memory section (corresponding to the “second memory section”) 109.

The frame memory section 104 stores the CFM frame data of the current frame representing the current scan. The persistence memory section 109 stores the CFM frame data of the previous frame that has been calculated and provided by the persistence computation section 108. The CFM frame data in the persistence memory section 109 has already been subjected to the persistence processing. In the following description, the blood flow velocity data of the CFM frame data that are stored in the frame memory section 104 and the persistence memory section 109 will be identified herein by Vcurrent and Vout−1, respectively.

The aliasing decision section 105 retrieves the blood flow velocity data Vcurrent and Vout−1 of the CFM frame data from the frame memory section 104 and the persistence memory section 109, respectively, and makes an aliasing decision based on those data. Specifically, the aliasing decision section 105 compares the blood flow velocity data Vcurrent and Vout−1 to multiple threshold values to determine whether or not aliasing has occurred and whether or not the blood flow velocity data Vcurrent is in an aliasing region. And the aliasing decision section 105 outputs the results to the persistence coefficient determining section 106 and the persistence computation section 108.

Based on the two decision results provided by the aliasing decision section 105 and on the blood flow velocity data Vcurrent that has been retrieved from the frame memory section 104, the persistence coefficient determining section 106 makes a reference index to the persistence coefficient reference memory section 107. Also, the persistence coefficient determining section 106 accesses the persistence coefficient reference memory section 107 to retrieve a persistence coefficient that is associated with the reference index and set the persistence coefficient with respect to the persistence computation section 108. In the persistence coefficient reference memory section 107, stored in advance is a table of reference of persistence coefficients that are associated with the blood flow velocity values. This table of reference includes at least two different values of persistence coefficients that are associated with blood flow velocity values.

Based on the persistence coefficient that has been set by the persistence coefficient determining section 106 and on the results of aliasing decision made by the aliasing decision section 105, the persistence computation section 108 performs a persistence computation represented by the following Equation (1) on the blood flow velocity data. Supposing the persistence-processed blood flow velocity data that has been obtained through the persistence computation is identified by Vout and the persistence coefficient is identified by Cpersistence (where 0<Cpersistence<1), the persistence-processed blood flow velocity data is calculated by the following Equation (1):

Vout=(1−Cpersistence)×Vcurrent+Cpersistence×Vout−1 (1)

If the CFM frame data includes data other than the blood flow velocity data, the persistence computation is performed using the respective data of the current and previous frames and the persistence coefficient Cpersistence calculated, thereby obtaining persistence-processed data.

If the result of the aliasing decision that has been made by the aliasing decision section 105 is true, then Equation (1) is treated as an unsigned arithmetic. On the other hand, if the result of the aliasing decision is false, then Equation (1) is treated as a signed arithmetic.

Since the measurement is supposed to be made using a pulse wave as described above, the blood flow velocity that can be measured directly with the Doppler shift is restricted by the pulse repetition frequency (PRF). Specifically, when aliasing occurs, blood flows, of which the velocities correspond to a frequency variation of more than ±PRF/2, are recognized by mistake as reverse blood flows.

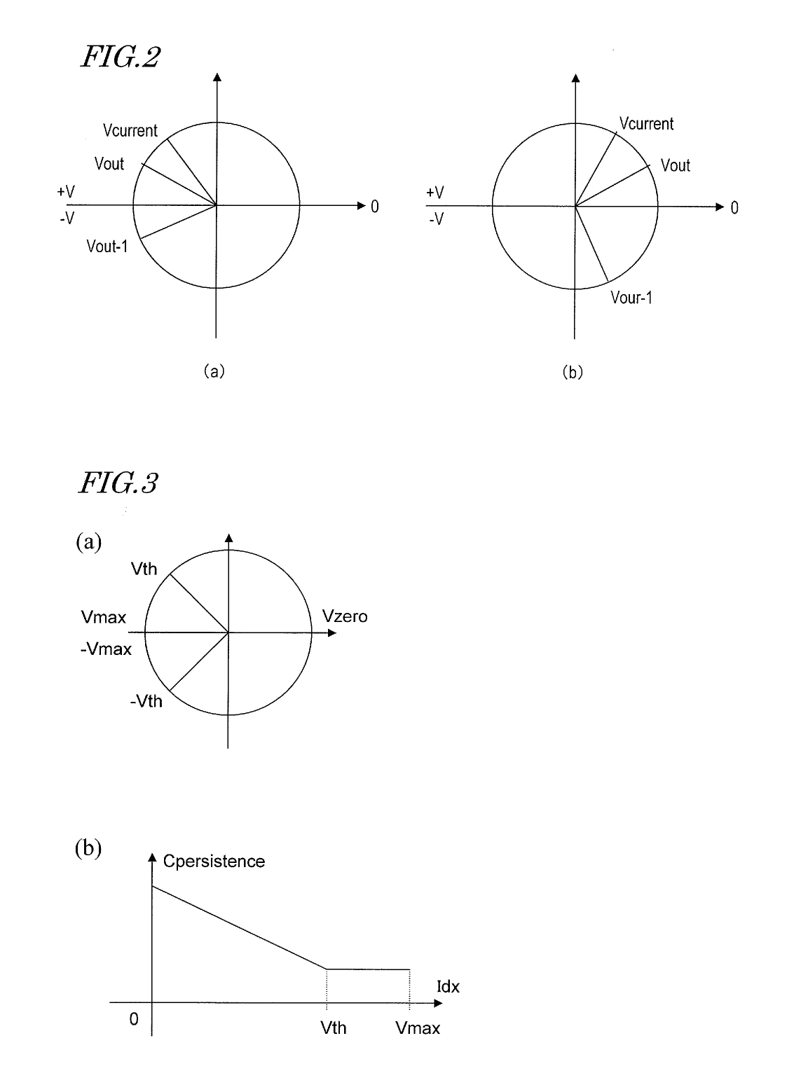

FIGS. 2(a) and 2(b) show the relation between the respective magnitudes of the persistence-processed blood flow velocity data Vout, the blood flow velocity data Vcurrent of the current frame, and the blood flow velocity data Vout−1 of the previous frame that has been provided by the persistence computation section 108. In FIGS. 2(a) and 2(b), the first quadrant on the axis of abscissas indicates a situation where the velocity V is zero, while the second quadrant thereof indicates a situation where the velocity V is either +V or −V. That is to say, a positive velocity V is located in the first or second quadrant, while a negative velocity V is located in the third or fourth quadrant.

For example, if Vcurrent and Vout−1 are located in the second and third quadrants, respectively, and if it has been determined that aliasing has occurred as shown in FIG. 2(a), Vout−1 actually becomes greater than a blood flow velocity corresponding to +PRF/2. That is why this is an arithmetic that does not cross zero, i.e., an unsigned arithmetic. For that reason, Vcurrent and Vout−1 may have their signs (+ or −) removed and their absolute values may be substituted into Equation (1) to make the arithmetic.

On the other hand, if Vcurrent and Vout−1 are located in the first and fourth quadrants, respectively, and if it has been determined that no aliasing has occurred as shown in FIG. 2(b), then Equation (1) is an arithmetic that does cross zero, i.e., a signed arithmetic. For that reason, Vcurrent and Vout−1 with signs may be substituted into Equation (1) to make the arithmetic. This arithmetic is performed on each pixel or measuring point of one frame of the blood flow velocity data. Also, if aliasing has occurred, Vout obtained as the result of the arithmetic becomes an unsigned value. In that case, if the most significant bit of the blood flow velocity data Vout is treated as a sign, Vout can be output as a signed value to the CFM DSC section 110 and the persistence memory section 109.

The CFM DSC section 110 converts the coordinates of the blood flow velocity data provided by the persistence computation section 108 and outputs the result to the image synthesizing section 113.

The tomographic image signal processing section 409 cuts down unwanted noise of the received signal by subjecting it to dynamic filtering and then subjects the received signal to envelope detection processing and dynamic range compression processing, thereby outputting tomographic image frame data to a tomographic image DSC section 410. In response, the tomographic image DSC section 410 converts the coordinates of the tomographic image frame data that has been provided by the tomographic image signal processing section 409 and then outputs the converted data to the image synthesizing section 411.

The image synthesizing section 411 synthesizes together the frame data that has been provided by the CFM DSC section 410 and the tomographic image DSC section 410 either on a pixel-by-pixel basis or on the data of each pair of associated measuring points, thereby generating synthesized image frame data. Specifically, the image synthesizing section 411 synthesizes together the two data either on a pixel-by-pixel basis or on the basis of each pair of associated measuring points so that if the blood flow velocity is zero, tomographic image frame data is displayed but that the CPM frame data is displayed otherwise. Also, the image synthesizing section 411 transforms the data into color information according to the blood flow velocity or blood flowing direction and then outputs the color information thus obtained to a display section 412, which displays the data provided by the image synthesizing section 411.

Next, it will be described in further detail how to determine the persistence coefficient. In order to determine the persistence coefficient, first, the aliasing decision section 105 needs to determine whether or not aliasing has occurred in the blood flow velocity.

The aliasing decision section 105 retrieves the blood flow velocity data Vcurrent of the current CFM frame data from the frame memory section 104 and the blood flow velocity data Vout−1 of the CFM frame data of the previous frame that has been calculated by the persistence computation section 108 from the persistence memory section 109, respectively. Then, based on the Vcurrent and Vout−1 values, the aliasing decision section 105 determines:
1. whether or not aliasing has occurred, and
2. whether or not Vcurrent is located in an aliasing region.

These two decisions are made by comparing Vcurrent and Vout−1 to a predetermined threshold value. Specifically, Vcurrent, Vout−1 and a zero blood flow velocity Vzero are compared to a threshold value Vth.

TABLE 1

| | | | Entered |
|---|---|---|---|
| | | Aliasing | aliasing |
| Condition | | occurred? | region? |
| (0) | If Vcurrent <− Vth and Vout − 1 > 0 | YES | YES |
| (1) | If Vcurrent > Vth and Vout − 1 < 0 | YES | YES |
| (2) | If Vout − 1 <− Vth and Vcurrent > 0 | YES | NO |
| (3) | If Vout − 1 > Vth and Vcurrent < 0 | YES | NO |
| (4) | Otherwise | NO | NO |

FIG. 3(a) shows the relation between the respective magnitudes of the threshold value Vth, the zero blood flow velocity Vzero, Vcurrent and Vout−1. In FIG. 3(a), the first quadrant on the axis of abscissas indicates a situation where the blood flow velocity V is the zero blood flow velocity Vzero, while the second quadrant thereof indicates a situation where the velocity V is either Vmax or −Vmax. That is to say, a positive velocity V is located in the first or second quadrant, while a negative velocity V is located in the third or fourth quadrant.

In this case, Vth and −Vth are set to be the maximum expected variation in blood flow velocity in a time interval between two consecutive frames.

Table 1 summarizes the condition to be examined by the aliasing decision section 105 and its decision results.

If Vout−1 is positive (i.e., if Condition (0) is satisfied), the maximum expected variation in blood flow velocity will be either Vth or −Vth. That is why Vcurrent is never less than −Vth. For that reason, as long as Vcurrent<−Vth is satisfied, Vcurrent is actually a greater value than the maximum blood flow velocity Vmax corresponding to +PRF/2. Consequently, it is determined that aliasing has occurred and that Vcurrent is in the aliasing region. Condition (1) is set by inverting the signs of Condition (0).

On the other hand, if Vout−1 is less than −Vth (i.e., if Condition (2) is satisfied), a positive Vcurrent value means that the variation in blood flow velocity that has occurred exceeds the maximum expected variation in blood flow velocity. That is why it is determined that aliasing has occurred. Also, since Vcurrent falls within the ±Vth range with Vzero interposed between them, Vcurrent is not in the aliasing region. Condition (3) is set by inverting the signs of Condition (2).

And if none of these Conditions (0) through (3) are satisfied, then it is determined that no aliasing has occurred and that Vcurrent is not located in the aliasing region.

Based on the two decision results provided by the aliasing decision section 105 and on the absolute value of the blood flow velocity data Vcurrent that has been retrieved from the frame memory section 104, the persistence coefficient determining section 106 makes a reference index to the persistence coefficient reference memory section 107. The reference indices made are shown in the following Table 2:

TABLE 2

| | | Entered | |
|---|---|---|---|
| | Aliasing | aliasing | Reference |
| Condition | occurred? | region? | index (Idx) |
| (0) | YES | YES | Vmax |
| (1) | YES | YES | Vmax |
| (2) | YES | NO | Abs (Vcurrent) |
| (3) | YES | NO | Abs (Vcurrent) |
| (4) | NO | NO | Abs (Vcurrent) |

If aliasing has occurred and if Vcurrent is in the aliasing region, the blood flow velocity Vcurrent should actually be even greater than Vmax or −Vmax. That is why the reference index becomes Vmax in that case. Otherwise, the reference index becomes the absolute value Abs(Vcurrent) of Vcurrent.

In the persistence coefficient reference memory section 107, stored in advance is a table of reference of persistence coefficients that are associated with the reference indices. Also, the persistence coefficient determining section 106 accesses the persistence coefficient reference memory section 107 to retrieve a persistence coefficient that is associated with the reference index made and output it to the persistence computation section 108.

FIG. 3(b) is a graph showing an exemplary correlation between the reference index and the persistence coefficient. In FIG. 3(b), the abscissa represents the reference index and the ordinate represents the persistence coefficient. As shown in Table 2, the reference index is either Vmax or the absolute value Abs(Vcurrent) of Vcurrent. If the absolute value of Vcurrent is equal to or smaller than the threshold value Vth, its associated persistence coefficient Cpersistence is defined so as to decrease monotonically as Vcurrent increases. In other words, if the absolute value of Vcurrent is equal to or smaller than the threshold value Vth, its associated persistence coefficient Cpersistence varies according to the blood flow velocity Vcurrent of the current frame. That is why if the blood flow velocity Vcurrent of the current frame is low, then the persistence coefficient Cpersistence is large. That is to say, the weight added to the blood flow velocity Vout−1 of the previous frame increases. As a result, if the blood flow velocity Vcurrent of the current frame is low, a blood flow velocity Vout, which depends heavily on the blood flow velocity Vout−1 of the previous frame, is determined and displayed on the display section 114. Consequently, the color flow mapping image changes smoothly and blackout arises much less often.

On the other hand, if the blood flow velocity Vcurrent of the current frame is high, then the persistence coefficient Cpersistence is small. That is to say, the weight added to the blood flow velocity Vout−1 of the previous frame decreases. As a result, if the blood flow velocity Vcurrent of the current frame is high, the influence of the blood flow velocity Vout−1 of the previous frame decreases. Consequently, a color flow mapping image, representing a steep increase in blood flow velocity in real time, is realized.

Furthermore, as Vcurrent increases, the persistence coefficient Cpersistence decreases monotonically. That is why if the blood flow velocity increases with time, the persistence coefficient Cpersistence decreases, the persistence effect declines, and the color flow mapping image changes more dramatically. On the other hand, if the blood flow velocity decreases with time, the persistence coefficient Cpersistence increases, so does the persistence effect, and the color flow mapping image changes more gently.

Also, as can be seen from Tables 1 and 2, even if Vcurrent<−Vth but if Vout−1>0, then the reference index becomes Vmax (i.e., Condition (0) is satisfied). Meanwhile, if Vout−1<0, then the reference index becomes the absolute value Abs(Vcurrent) of Vcurrent (i.e., Condition (4) is satisfied). That is why even if Vcurrent<−Vth is satisfied, both the reference index and the persistence coefficient Cpersistence change depending on whether Vout−1 is positive or negative. Consequently, even if both of two adjacent regions satisfy Vcurrent<−Vth, the color flow mapping image displayed changes its colors according to the sign of Vout−1. As a result, the image displayed comes to have portions where the color tone changes discontinuously.

To avoid displaying such an unnatural image, if the absolute value of Vcurrent is equal to or greater than the threshold value Vth, then it is preferred that every reference index be associated with a persistence coefficient Cpersistence of the same value. In that case, even a blood flow region where aliasing has occurred and its surrounding regions can also be displayed as natural images.

As described above, the ultrasonic diagnostic apparatus of this preferred embodiment determines a persistence coefficient for the CFM frame data dynamically according to the blood flow velocity and the status of aliasing, and then performs the persistence computation. As a result, this ultrasonic diagnostic apparatus can accurately sense any variation in blood flow even at any region to inspect where the blood flow velocity changes significantly (such as a carotid artery) and can display a blood flow moving picture smoothly without causing any blackout even at a low blood flow velocity.

In the preferred embodiment described above, the persistence coefficient is supposed to be determined dynamically according to the blood flow velocity of the CFM frame data and perform the persistence computation on the blood flow velocity. However, the persistence computation may also be performed on other data of the CFM frame data (such as the blood flow power data described above). Or the persistence computation could also be performed on B-mode tomographic image data.

Also, in the preferred embodiment described above, the persistence processing is supposed to be carried out using the respective blood flow velocity data of the current frame and the previous frame. However, this is just an example of the present invention. Alternatively, the persistence processing may also be carried out using the blood flow velocity data of the frame before the previous frame or an even earlier frame. Furthermore, the persistence processing does not always have to be performed using Equation (1) but may also be performed using any other equation.

### Embodiment 2

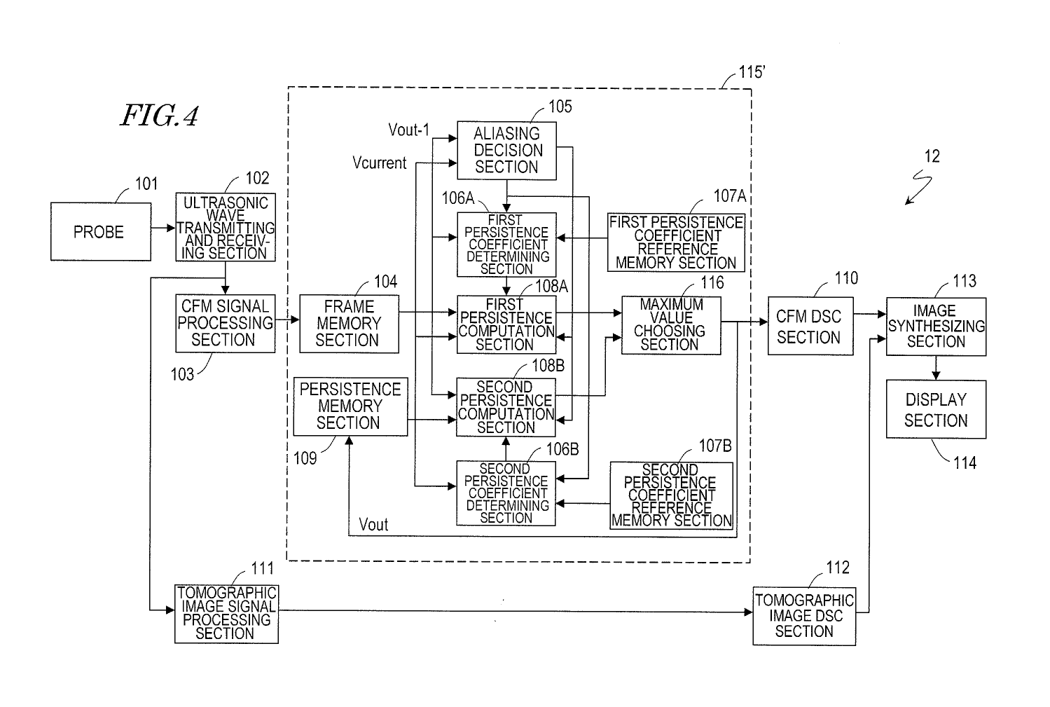

Hereinafter, a second preferred embodiment of an ultrasonic diagnostic apparatus according to the present invention will be described with reference to the accompanying drawings. FIG. 4 is a block diagram illustrating a second preferred embodiment of an ultrasonic diagnostic apparatus according to the present invention. The ultrasonic diagnostic apparatus 12 shown in FIG. 4 includes a probe 101, an ultrasonic wave transmitting and receiving section 102, a CFM signal processing section 103, a persistence processing 115′, a tomographic image signal processing section 111, a CFM DSC section 110, a tomographic image DSC section 112, an image synthesizing section 113, and a display section 114. Among these components, the probe 101 and the display section 114 may be general-purpose ones and may be omitted from this ultrasonic diagnostic apparatus 12.

As already described for the first preferred embodiment, the ultrasonic wave transmitting and receiving section 102 generates a drive signal to drive the probe 101 and outputs the signal to the probe 101, thereby sending out an ultrasonic wave from the probe 101 toward a subject. Also, the ultrasonic wave transmitting and receiving section 102 makes the probe 101 receive reflected echoes, which have been produced by having the transmitted ultrasonic wave reflected by the subject, thereby generating a received signal. More specifically, the probe 101 is made up of multiple piezoelectric transducers. And this ultrasonic wave transmitting and receiving section 102 drives the probe 101 while controlling the delays caused by those piezoelectric transducers so that the ultrasonic wave sent out by each of those piezoelectric transducers defines a single ultrasonic beam and that the subject is scanned with a number of such ultrasonic beams. Those reflected echoes are received by the respective piezoelectric transducers and the ultrasonic wave transmitting and receiving section 102 controls the delays caused by the respective piezoelectric transducers, thereby generating received signals that are associated with the respective ultrasonic beams transmitted. In this case, every time the subject is scanned with an ultrasonic beam, data for one frame can be obtained. And by repeatedly transmitting and receiving ultrasonic waves several to several tens of times a second, several to several tens of frames of received signals are generated one after another per second.

The ultrasonic diagnostic apparatus 12 of this preferred embodiment generates a B-mode tomographic image and a color flow mapping image, synthesizes these two images together, and then displays the synthetic image on the display section 114. Thus, the ultrasonic wave transmitting and receiving section 102 transmits and receives ultrasonic waves both in order to generate the B-mode tomographic image and to generate the color flow mapping image. The B-mode tomographic image and the color flow mapping image may or may not be displayed at the same frame rate (i.e., may or may not have the same number of frames to display per second). If these two kinds of images have the same frame rate, ultrasonic waves may be alternately transmitted and received to generate a B-mode tomographic image and to generate a color flow mapping image.

In generating a B-mode tomographic image, the ultrasonic wave transmitting and receiving section 102 controls its mode of transmitting and receiving ultrasonic waves appropriately so as to generate the B-mode tomographic image as intended and outputs a received signal obtained to the tomographic image signal processing section 111. On the other hand, in generating a color flow mapping tomographic image, the ultrasonic wave transmitting and receiving section 102 controls its mode of transmitting and receiving ultrasonic waves appropriately so as to generate the color flow mapping tomographic image as intended and outputs a received signal obtained to the CFM signal processing section 103. In general, when a color flow mapping tomographic image needs to be generated, the ultrasonic wave transmitting and receiving section 102 repeatedly transmits and receives ultrasonic waves a number of times along the same acoustic line to generate a color flow mapping tomographic image with good stability.

The CFM signal processing section 103 performs orthogonal detection processing, MTI filtering and autocorrelation processing on the received signal to calculate a blood flow velocity and a blood flow power, and then carries out noise reduction processing to cut down either system noise or acoustic noise. The CFM frame data includes at least blood flow velocity data but may also include blood flow power data and blood flow velocity variance data as well. The CFM signal processing section 103 repeatedly performs this processing on each of the received signals that form respective frames one after another. The CFM frame data generated by the CFM signal processing section 103 is output to the persistence processing section 115′ on a frame-by-frame basis.

The persistence processing section 115′ performs persistence processing on the CFM frame data on a frame-by-frame basis using a persistence coefficient. The ultrasonic diagnostic apparatus 12 of this preferred embodiment determines the persistence coefficient by the blood flow velocity. That is to say, the persistence coefficient is not a constant value but is a dynamic value that is variable according to the blood flow velocity. Consequently, the persistence coefficient can be changed, and the persistence effect can be controlled, according to the blood flow velocity. To present the blood flow as a moving picture, however, ultrasonic waves need to be transmitted and received with a pulse Doppler system. That is why the measurable blood flow velocity is restricted by the pulse repetition frequency (PRF). As a result, aliasing will occur in the blood flow velocity, thus making it difficult to estimate the blood flow velocity accurately.

To determine whether aliasing has occurred or not, the ultrasonic diagnostic apparatus 12 of this preferred embodiment uses the respective blood flow velocity data of the current frame and the previous frame. For that purpose, the persistence processing section 115′ includes two persistence computation sections for performing simultaneously a first persistence computation to change the blood flow velocity quickly without producing so much persistence effect and a second persistence computation to keep the variation in blood flow velocity as small as possible with a lot of persistence effect generated, respectively. Two blood flow velocity data, which will produce different degrees of persistence effect, are obtained. And by using one of two data that has the greater absolute value, the persistence processing section 115′ generates a blood flow image. As a result, even the blood flowing through a peripheral blood vessel, where the blood flow has low power, can be displayed constantly without flickering. And a blood flow moving picture can be displayed consistently without letting an overly smoothed blood, flowing through the peripheral blood vessel, disappear from the screen.

For that purpose, the persistence processing section 115′ includes a frame memory section (corresponding to the “first memory section”) 104, an aliasing decision section 105, a first persistence coefficient determining section 106A, a first persistence coefficient reference memory section (corresponding to the “third memory section”) 107A, a first persistence computation section 108A, a second persistence coefficient determining section 106B, a second persistence coefficient reference memory section (corresponding to the “fourth memory section”) 107B, a second persistence computation section 108B, a maximum value choosing section 116 and a persistence memory section (corresponding to the “second memory section”) 109.

The frame memory section 104 stores the CFM frame data of the current frame representing the current scan. The persistence memory section 109 stores the CFM frame data of the previous frame that has been calculated and provided by the maximum value choosing section. The CFM frame data in the persistence memory section 109 has already been subjected to the persistence processing. As in the first preferred embodiment described above, the blood flow velocity data of the CFM frame data that are stored in the frame memory section 104 and the persistence memory section 109 will be identified herein by Vcurrent and Vout−1, respectively.

The aliasing decision section 105 retrieves the blood flow velocity data Vcurrent and Vout−1 of the CFM frame data from the frame memory section 104 and the persistence memory section 109, respectively, and makes an aliasing decision based on those data. Specifically, the aliasing decision section 105 compares the blood flow velocity data Vcurrent and Vout−1 to multiple threshold values to determine whether or not aliasing has occurred and whether or not the blood flow velocity data Vcurrent is in an aliasing region. And the aliasing decision section 105 outputs the results to the first and second persistence coefficient determining sections 106A and 106B and the first and second persistence computation sections 108A and 108B.

Based on the two decision results provided by the aliasing decision section 105 and on the blood flow velocity data Vcurrent that has been retrieved from the frame memory section 104, the first persistence coefficient determining section 106A makes a reference index to the first persistence coefficient reference memory section 107A. Also, the first persistence coefficient determining section 106A accesses the first persistence coefficient reference memory section 107A to retrieve a first persistence coefficient that is associated with the reference index and set the first persistence coefficient with respect to the first persistence computation section 108A. In the first persistence coefficient reference memory section 107A, stored in advance is a first table of reference of a first set of persistence coefficients that are associated with the blood flow velocity values. This first table of reference includes at least two different values of persistence coefficients that are associated with blood flow velocity values.

On the other hand, based on the two decision results provided by the aliasing decision section 105 and on the blood flow velocity data Vout−1 that has been retrieved from the persistence memory section 109, the second persistence coefficient determining section 106B makes a reference index to the second persistence coefficient reference memory section 107B. Also, the second persistence coefficient determining section 106B accesses the second persistence coefficient reference memory section 107B to retrieve a second persistence coefficient that is associated with the reference index and set the second persistence coefficient with respect to the second persistence computation section 108B. In the second persistence coefficient reference memory section 107B, stored in advance is a second table of reference of a second set of persistence coefficients that are associated with the blood flow velocity values. This second table of reference also includes at least two different values of persistence coefficients that are associated with blood flow velocity values. As will be described in detail later, even if one of the persistence coefficients of the first set and one of the persistence coefficients of the second set are associated with the same blood flow velocity value, those two persistence coefficients actually have mutually different values.

Based on the persistence coefficient that has been set by the first persistence coefficient determining section 106A and on the results of aliasing decision made by the aliasing decision section 105, the first persistence computation section 108A performs a persistence computation represented by the following Equation (1) on the blood flow velocity data.

Supposing the persistence-processed blood flow velocity data that has been obtained through the persistence computation is identified by Vout and the persistence coefficient is identified by Cpersistence (where 0<Cpersistence<1), the persistence-processed blood flow velocity data is calculated by the following Equation (1):

Vout=(1−Cpersistence)×Vcurrent+Cpersistence×Vout−1 (1)

In the same way, based on the persistence coefficient that has been set by the second persistence coefficient determining section 106B and on the results of aliasing decision made by the aliasing decision section 105, the second persistence computation section 108B performs a persistence computation represented by Equation (1) on the blood flow velocity data.

The computations performed by the first and second persistence computation sections 108A and 108B are the same except that the persistence coefficients determined are different from each other. If the CFM frame data includes data other than the blood flow velocity data, the first and second persistence computation sections 108A and 108B perform the persistence computations using the respective data of the current and previous frames and the persistence coefficient Cpersistence calculated, thereby obtaining persistence-processed data.

If the result of the aliasing decision that has been made by the aliasing decision section 105 is true, then Equation (1) is treated as an unsigned arithmetic. On the other hand, if the result of the aliasing decision is false, then Equation (1) is treated as a signed arithmetic.

Since the measurement is supposed to be made using a pulse wave as described above, the blood flow velocity that can be measured directly with the Doppler shift is restricted by the pulse repetition frequency (PRF). Specifically, when aliasing occurs, blood flows, of which the velocities correspond to a frequency variation of more than ±PRF/2, are recognized by mistake as reverse blood flows.

FIGS. 5(a) and 5(b) show the relation between the respective magnitudes of the persistence-processed blood flow velocity data Vout, the blood flow velocity data Vcurrent of the current frame, and the blood flow velocity data Vout−1 of the previous frame that has been provided by the persistence computation section 108. In FIGS. 5(a) and 5(b), the first quadrant on the axis of abscissas indicates a situation where the velocity V is zero, while the second quadrant thereof indicates a situation where the velocity V is either +V or −V. That is to say, a positive velocity V is located in the first or second quadrant, while a negative velocity V is located in the third or fourth quadrant.

For example, if Vcurrent and Vout−1 are located in the second and third quadrants, respectively, and if it has been determined that aliasing has occurred as shown in FIG. 5(a), Vout−1 actually becomes greater than a blood flow velocity corresponding to +PRF/2. That is why this is an arithmetic that does not cross zero, i.e., an unsigned arithmetic. For that reason, Vcurrent and Vout−1 may have their signs (+ or −) removed and their absolute values may be substituted into Equation (1) to make the arithmetic.

On the other hand, if Vcurrent and Vout−1 are located in the first and fourth quadrants, respectively, and if it has been determined that no aliasing has occurred as shown in FIG. 5(b), then Equation (1) is an arithmetic that does cross zero, i.e., a signed arithmetic. For that reason, Vcurrent and Vout−1 with signs may be substituted into Equation (1) to make the arithmetic. This arithmetic is performed on each pixel or measuring point of one frame of the blood flow velocity data. Also, if aliasing has occurred, Vout obtained as the result of the arithmetic becomes an unsigned value. In that case, if the most significant bit of the blood flow velocity data Vout is treated as a sign, Vout can be output as a signed value to the maximum value choosing section 116.

The maximum value choosing section 116 receives the results of the arithmetic, i.e., the persistence-processed blood flow velocity data, from the first and second persistence computation sections 108A and 108B, compares the absolute values of the blood flow velocities either on a pixel-by-pixel basis or on the data of each pair of associated measuring points, chooses the greater blood flow velocity data as the persistence-processed blood flow velocity data of the current frame, and outputs it to the CFM DSC section 110 and the persistence memory section 109. The CFM DSC section 110 converts the coordinates of the blood flow velocity data chosen and outputs the result to the image synthesizing section 113.

The tomographic image signal processing section 409 cuts down unwanted noise of the received signal by subjecting it to dynamic filtering and then subjects the received signal to envelope detection processing and dynamic range compression processing, thereby outputting tomographic image frame data to a tomographic image DSC section 410. In response, the tomographic image DSC section 410 converts the coordinates of the tomographic image frame data that has been provided by the tomographic image signal processing section 409 and then outputs the converted data to the image synthesizing section 411.

The image synthesizing section 411 synthesizes together the frame data that has been provided by the CFM DSC section 410 and the tomographic image DSC section 410 either on a pixel-by-pixel basis or on the data of each pair of associated measuring points, thereby generating synthesized image frame data. Specifically, the image synthesizing section 411 synthesizes together the two data either on a pixel-by-pixel basis or on the basis of each pair of associated measuring points so that if the blood flow velocity is zero, tomographic image frame data is displayed but that the CFM frame data is displayed otherwise. Also, the image synthesizing section 411 transforms the data into color information according to the blood flow velocity or blood flowing direction and then outputs the color information thus obtained to a display section 412, which displays the data provided by the image synthesizing section 411.

Next, it will be described in further detail how to determine the first and second persistence coefficients. In order to determine the first and second persistence coefficients, first, the aliasing decision section 105 needs to determine whether or not aliasing has occurred in the blood flow velocity.

The aliasing decision section 105 retrieves the blood flow velocity data Vcurrent of the current CFM frame data from the frame memory section 104 and the blood flow velocity data Vout−1 of the CFM frame data of the previous frame that has been calculated by the persistence computation section 108 from the persistence memory section 109, respectively. Then, based on the Vcurrent and Vout−1 values, the aliasing decision section 105 determines:
1. whether or not aliasing has occurred, and
2. whether or not Vcurrent is located in an aliasing region.

These two decisions are made by comparing Vcurrent and Vout−1 to a predetermined threshold value. Specifically, Vcurrent, Vout−1 and a zero blood flow velocity Vzero are compared to a threshold value Vth.

TABLE 3

| | | | Entered |
|---|---|---|---|
| | | Aliasing | aliasing |
| Condition | | occurred? | region? |
| (0) | If Vcurrent <− Vth and Vout − 1 > 0 | YES | YES |
| (1) | If Vcurrent > Vth and Vout − 1 < 0 | YES | YES |
| (2) | If Vout − 1 <− Vth and Vcurrent > 0 | YES | NO |
| (3) | If Vout − 1 > Vth and Vcurrent < 0 | YES | NO |
| (4) | Otherwise | NO | NO |

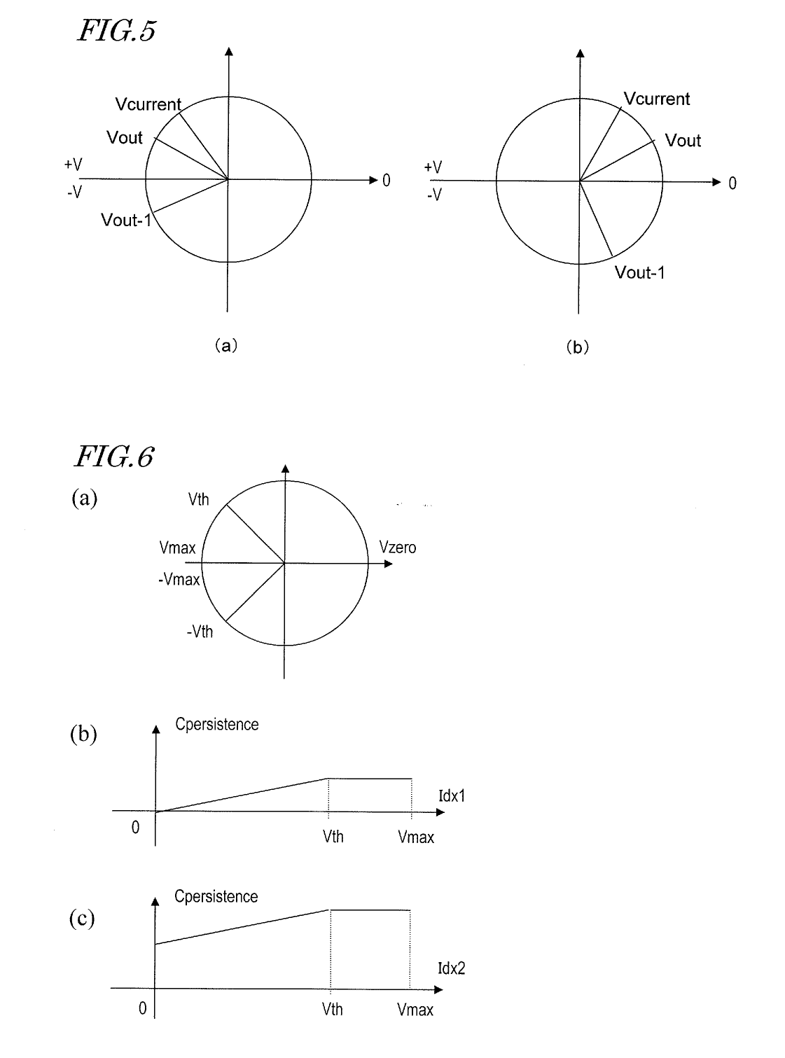

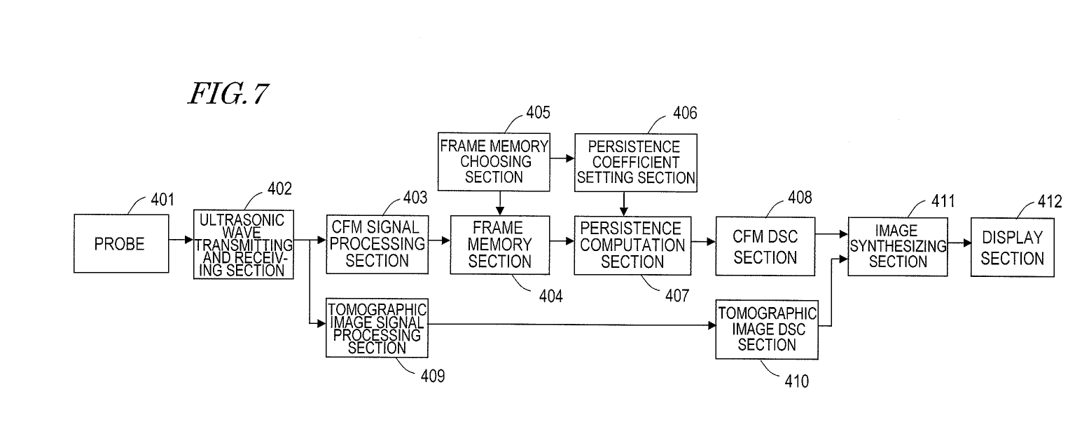

FIG. 6(a) shows the relation between the respective magnitudes of the threshold value Vth, the zero blood flow velocity Vzero, Vcurrent and Vout−1. In FIG. 6(a), the first quadrant on the axis of abscissas indicates a situation where the blood flow velocity V is the zero blood flow velocity Vzero, while the second quadrant thereof indicates a situation where the velocity V is either Vmax or −Vmax. That is to say, a positive velocity V is located in the first or second quadrant, while a negative velocity V is located in the third or fourth quadrant.

In this case, Vth and −Vth are set to be the maximum expected variation in blood flow velocity in a time interval between two consecutive frames.

Table 3 summarizes the condition to be examined by the aliasing decision section 105 and its decision results.

If Vout−1 is positive (i.e., if Condition (0) is satisfied), the maximum expected variation in blood flow velocity will be either Vth or −Vth. That is why Vcurrent is never less than −Vth. For that reason, as long as Vcurrent<−Vth is satisfied, Vcurrent is actually a greater value than the maximum blood flow velocity Vmax corresponding to +PRF/2. Consequently, it is determined that aliasing has occurred and that Vcurrent is in the aliasing region. Condition (1) is set by inverting the signs of Condition (0).

On the other hand, if Vout−1 is less than −Vth (i.e., if Condition (2) is satisfied), a positive Vcurrent value means that the variation in blood flow velocity that has occurred exceeds the maximum expected variation in blood flow velocity. That is why it is determined that aliasing has occurred. Also, since Vcurrent falls within the ±Vth range with Vzero interposed between them, Vcurrent is not in the aliasing region. Condition (3) is set by inverting the signs of Condition (2).

And if none of these Conditions (0) through (3) are satisfied, then it is determined that no aliasing has occurred and that Vcurrent is not located in the aliasing region.

Based on the two decision results provided by the aliasing decision section 105 and on the absolute value of the blood flow velocity data Vcurrent that has been retrieved from the frame memory section 104, the first persistence coefficient determining section 106A makes a reference index to the first persistence coefficient reference memory section 107. The reference indices made are shown in the following Table 4:

TABLE 4

| | | Entered | |
|---|---|---|---|
| | Aliasing | aliasing | Reference |
| Condition | occurred? | region? | index (Idx1) |
| (0) | YES | YES | Vmax |
| (1) | YES | YES | Vmax |
| (2) | YES | NO | Abs (Vcurrent) |
| (3) | YES | NO | Abs (Vcurrent) |
| (4) | NO | NO | Abs (Vcurrent) |

If aliasing has occurred and if Vcurrent is in the aliasing region, the blood flow velocity Vcurrent should actually be even greater than Vmax or −Vmax. That is why the reference index becomes Vmax in that case. Otherwise, the reference index becomes the absolute value Abs(Vcurrent) of Vcurrent.

In the first persistence coefficient reference memory section 107A, stored in advance is a first table of reference of a first set of persistence coefficients that are associated with the reference indices. Also, the first persistence coefficient determining section 106A accesses the first persistence coefficient reference memory section 107A to retrieve one of the persistence coefficients of the first set that is associated with the reference index made and output it to the first persistence computation section 108A.

FIG. 6(b) is a graph showing an exemplary correlation between the reference index and the first set of persistence coefficients. In FIG. 6(b), the abscissa represents the reference index and the ordinate represents the persistence coefficient. As shown in Table 4, the reference index is either Vmax or the absolute value Abs(Vcurrent) of Vcurrent. If the absolute value of Vcurrent is equal to or smaller than the threshold value Vth, its associated persistence coefficient Cpersistence of the first set is defined so as to decrease monotonically as Vcurrent increases. In other words, if the absolute value of Vcurrent is equal to or smaller than the threshold value Vth, its associated persistence coefficient Cpersistence varies according to the blood flow velocity Vcurrent of the current frame.

On the other hand, based on the two decision results provided by the aliasing decision section 105 and on the absolute value of the blood flow velocity data Vout−1 that has been retrieved from the persistence memory section 109, the second persistence coefficient determining section 106B makes a reference index to the first persistence coefficient reference memory section 107. The reference indices made are shown in the following Table 5:

TABLE 5

| | | Entered | |
|---|---|---|---|
| | Aliasing | aliasing | Reference |
| Condition | occurred? | region? | index (Idx2) |
| (0) | YES | YES | Vmax |
| (1) | YES | YES | Vmax |
| (2) | YES | NO | Abs (Vout − 1) |
| (3) | YES | NO | Abs (Vout − 1) |
| (4) | NO | NO | Abs (Vout − 1) |

Unlike the first persistence coefficient determining section 106A, the second persistence coefficient determining section 106B regards the absolute value of the blood flow velocity data Vout−1 that has been retrieved from the persistence memory section 109 as a reference index.

In the second persistence coefficient reference memory section 107B, stored in advance is a second table of reference of a second set of persistence coefficients that are associated with the reference indices. Also, the second persistence coefficient determining section 106B accesses the second persistence coefficient reference memory section 107B to retrieve one of the persistence coefficients of the second set that is associated with the reference index made and output it to the second persistence computation section 108B.

FIG. 6(c) is a graph showing an exemplary correlation between the reference index and the second set of persistence coefficients. In FIG. 6(c), the abscissa represents the reference index and the ordinate represents the persistence coefficient. As shown in Table 5, the reference index is either Vmax or the absolute value Abs(Vout−1) of Vout−1. If the absolute value of Vout−1 is equal to or smaller than the threshold value Vth, its associated persistence coefficient Cpersistence of the second set is defined so as to increase monotonically as Vout−1 increases. In other words, if the absolute value of Vout−1 is equal to or smaller than the threshold value Vth, its associated persistence coefficient Cpersistence of the second set varies according to the blood flow velocity Vout−1 of the previous frame.

As shown in FIGS. 6(b) and 6(c), no matter what value the reference index has, each persistence coefficient of the second set is always greater than its associated persistence coefficient of the first set. Specifically, each persistence coefficient of the first set is associated with the blood flow velocity of the current frame and has a small value. If the persistence coefficient of the first set increases, then the computation will be performed depending more heavily on the blood flow velocity of the previous frame. That is why the first persistence computation section 108A performs a computation so as to change the blood flow velocity quickly by decreasing the persistence effect. On the other hand, each persistence coefficient of the second set is associated with the blood flow velocity of the previous frame and has a large value. That is why the second persistence computation section 108B performs a computation so as to reduce the variation in blood flow velocity by increasing the persistence effect.

Also, the first persistence computation section 108A performs a computation so as to change the blood flow velocity quickly by decreasing the persistence effect as described above. That is why in a situation where the blood flow velocity is high but the blood flow power is too low to detect the blood flow properly, the blood flow velocity may suddenly go zero. In that case, if the blood flow image keeps on being colored in either tones or grayscales as the blood flow velocity goes higher and higher, then the blood flow image may be suddenly colored in a dark tone and start flickering on the screen. That is why by increasing monotonically the persistence coefficient of the first set as the reference index increases, the persistence effect can be increased and that flickering of the blood flow image can be minimized even as the blood flow velocity increases.

On the other hand, the second persistence computation section 108B contributes to displaying an image with the persistence effect increased. That is why in a situation where the blood flow image keeps on being colored in either tones or grayscales as the blood flow velocity goes higher and higher, if the blood flow velocity is too low, a relatively dark image will be displayed as persistence for an unnecessarily long time. In that case, if the probe is moved, the viewer will find the blood flow displayed for an excessively long time, for example. That is why by increasing monotonically the persistence coefficient of the second set as the reference index increases, the persistence effect can be decreased as the blood flow velocity decreases. Consequently, by defining appropriate monotonically increasing relation between the reference index based on the absolute value of the blood flow velocity and the first and second persistence coefficients, blood flow can be displayed with high image quality.

Also, as can be seen from Tables 3, 4 and 5, even if Vcurrent<−Vth but if Vout−1>0, then the reference index becomes Vmax (i.e., Condition (0) is satisfied). Meanwhile, if Vout−1<0, then the reference index becomes the absolute value Abs(Vcurrent) of Vcurrent (i.e., Condition (4) is satisfied). That is why even if Vcurrent<−Vth is satisfied, both the reference index and the persistence coefficient Cpersistence change depending on whether Vout−1 is positive or negative. Consequently, even if both of two adjacent regions satisfy Vcurrent<−Vth, the color flow mapping image displayed changes its colors according to the sign of Vout−1. As a result, the image displayed comes to have portions where the color tone changes discontinuously.

To avoid displaying such an unnatural image, if the absolute value of Vcurrent is equal to or greater than the threshold value Vth, then it is preferred that every reference index be associated with a persistence coefficient Cpersistence of the same value. In that case, even a blood flow region where aliasing has occurred and its surrounding regions can also be displayed as natural images.

Using these persistence coefficients of the first and second sets that have been determined as described above, the first and second persistence computation sections 108A and 108B generate latest blood flow velocity data that has been subjected to the persistence processing.

The maximum value choosing section 116 chooses one of the two blood flow velocity data that has the greater absolute value and outputs the blood flow velocity data thus chosen as persistence-processed blood flow velocity data. In other words, one of the two results of the persistence processing is chosen so as to obtain blood flow velocity data with the greater absolute value. Consequently, even though a thyroid, a liver, a kidney and other organs have a peripheral vessel where the blood flow power often loses stability, the blood flowing through the peripheral vessel can also be displayed without flickering. In addition, a blood flow moving picture can be displayed without smoothing out the blood flowing through the peripheral blood vessel too much to keep the peripheral blood vessel visible.

In the preferred embodiments described above, the persistence coefficient is determined dynamically by the blood flow velocity of the CFM frame data and the persistence computation is performed on that blood flow velocity. However, this is just an example of the present invention. Alternatively, the persistence computation may also be performed on blood flow power data or any data other than the CFM frame data and may even be performed on B-mode tomographic image data.

Also, in the preferred embodiments described above, the persistence processing is supposed to be performed using the blood flow velocity data of the current and previous frames. However, the persistence processing may also be performed using the blood flow velocity data of the frame before the previous frame or an even earlier frame. Also, the persistence processing may also be carried out by arithmetic expression other than Equation (1).

### INDUSTRIAL APPLICABILITY

The ultrasonic diagnostic apparatus of the present invention can be used particularly effectively to display a blood flow status of a subject.

### REFERENCE SIGNS LIST

101, 401 probe
102, 402 ultrasonic wave transmitting and receiving section
103, 403 CFM signal processing section
104, 404 frame memory section
105 aliasing decision section
106 persistence coefficient determining section
106A first persistence coefficient determining section
106B second persistence coefficient determining section
107 persistence coefficient reference memory section
107A first persistence coefficient reference memory section
107B second persistence coefficient reference memory section
108, 407 persistence computation section
108A first persistence computation section
108B second persistence computation section
109 persistence memory section
110, 408 CFM DSC section
111, 409 tomographic image signal processing section
112, 410 tomographic image DSC section
113, 411 image synthesizing section
114, 412 display section
115, 115′ persistence processing section
116 maximum value choosing section
405 frame memory selecting section
406 persistence coefficient setting section

1. An ultrasonic diagnostic apparatus comprising:
a transmitting and receiving section, which drives a probe a number of times to send out ultrasonic waves toward a subject and which makes the probe receive reflected echoes that have been produced by having the ultrasonic waves reflected by the subject, thereby generating multiple received signals one after another;
a color flow mapping signal processing section for sequentially generating, based on the received signals, blood flow velocity data about a portion of each frame representing the blood flow of the subject;
a persistence processing section for performing persistence processing on the blood flow velocity data of each said frame;
a tomographic image signal processing section for generating B-mode tomographic image frame data based on the received signals; and
an image synthesizing section for synthesizing together the persistence-processed blood flow velocity data and the B-mode tomographic image frame data,
wherein the persistence processing section makes an aliasing decision based on the blood flow velocity data of the current frame and the persistence-processed blood flow velocity data of an earlier frame that precedes the current frame, and changes a persistence coefficient dynamically according to a result of the aliasing decision and based on those blood flow velocity data of the current and earlier frames.

2. The ultrasonic diagnostic apparatus of claim 1, wherein the persistence processing section includes:
a first memory section for storing the blood flow velocity data of the current frame;
a second memory section for storing the persistence-processed blood flow velocity data of the earlier frame that precedes the current frame;
an aliasing decision section for making an aliasing decision by retrieving the respective blood flow velocity data from the first and second memory sections;
a persistence coefficient determining section for determining the persistence coefficient based on a result of the aliasing decision and on the blood flow velocity data that is stored in the first memory section; and
a persistence computation section for performing a persistence computation on the blood flow velocity data that is stored in the first memory section using the persistence coefficient based on the result of the aliasing decision and outputting a result of the computation as the persistence-processed blood flow velocity data.

3. The ultrasonic diagnostic apparatus of claim 2, wherein by comparing the respective blood flow velocity data that are stored in the first and second memory sections to multiple threshold values, the aliasing decision section determines whether or not aliasing has occurred and whether or not the blood flow velocity data of the current frame is in an aliasing region.

4. The ultrasonic diagnostic apparatus of claim 2, wherein the persistence processing section further includes a third memory section that stores a table of reference including persistence coefficients with two or more different values that are associated with the blood flow velocity values.

5. The ultrasonic diagnostic apparatus of claim 4, wherein in the table of reference, a persistence coefficient with a constant value is associated with blood flow velocities, of which the values are equal to or greater than a predetermined value.

6. The ultrasonic diagnostic apparatus of claim 1, wherein the persistence processing section includes:
a first memory section for storing the blood flow velocity data of the current frame;
a second memory section for storing the persistence-processed blood flow velocity data of the earlier frame that precedes the current frame;
an aliasing decision section for making an aliasing decision by retrieving the respective blood flow velocity data from the first and second memory sections;
a first persistence coefficient determining section for determining a first persistence coefficient based on a result of the aliasing decision and on the blood flow velocity data that is stored in the first memory section;
a first persistence computation section for performing a persistence computation on the blood flow velocity data that is stored in the first memory section using the first persistence coefficient based on the result of the aliasing decision;
a second persistence coefficient determining section for determining a second persistence coefficient based on the result of the aliasing decision and on the blood flow velocity data that is stored in the second memory section;
a second persistence computation section for performing a persistence computation on the blood flow velocity data that is stored in the first memory section using the second persistence coefficient based on the result of the aliasing decision; and
a maximum value choosing section for comparing the two absolute values of the computational results provided by the first and second persistence computation sections to each other and outputting the greater one of the two absolute values as the persistence-processed blood flow velocity data.

7. The ultrasonic diagnostic apparatus of claim 6, wherein by comparing the respective blood flow velocity data that are stored in the first and second memory sections to multiple threshold values, the aliasing decision section determines whether or not aliasing has occurred and whether or not the blood flow velocity data of the current frame is in an aliasing region.

8. The ultrasonic diagnostic apparatus of claim 6, wherein the persistence processing section further includes:
a third memory section that stores a first table of reference including a first set of persistence coefficients with two or more different values that are associated with the blood flow velocity values; and
a fourth memory section that stores a second table of reference including a second set of persistence coefficients with two or more different values that are associated with the blood flow velocity values.

9. The ultrasonic diagnostic apparatus of claim 8, wherein even if one of the persistence coefficients of the first set and one of the persistence coefficients of the second set, which are stored in the first and second tables of reference, respectively, are associated with the same blood flow velocity value, those two persistence coefficients have mutually different values.

10. The ultrasonic diagnostic apparatus of claim 8, wherein in the first table of reference, a persistence coefficient with a constant value is associated with blood flow velocities, of which the values are equal to or greater than a predetermined value.

11. The ultrasonic diagnostic apparatus of claim 1, wherein the persistence-processed blood flow velocity data of the earlier frame that precedes the current frame belongs to the previous frame.
